SEQUENCE LISTING (6 sequences)

SEQ ID NO: 1 - DNA, 1191 bp, Escherichia coli
  CDS (1)..(1191)
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaa

SEQ ID NO: 2 - protein, 396 aa, Escherichia coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 3 - DNA, 36 bp, Artificial Sequence
  Description of Artificial Sequence Primer
gctacttacgaattccgtttgtcatcagtctcagcc

SEQ ID NO: 4 - DNA, 36 bp, Artificial Sequence
  Description of Artificial Sequence Primer
cctagatcacagctgatgtttgagaacattaccgcc

SEQ ID NO: 5 - DNA, 36 bp, Artificial Sequence
  Description of Artificial Sequence Synthetic oligonucleotide
ttgactattttacctctggcggtgataatggtccca

SEQ ID NO: 6 - DNA, 40 bp, Artificial Sequence
  Description of Artificial Sequence Synthetic oligonucleotide
agcttgggaccattatcaccgccagaggtaaaatagtcaa

(19) United States
(12) Patent Application Publication
(10) Pub. No.: US 2004/0132165 A1
(43) Pub. Date: Jul. 8, 2004

(54) Method for producing L-threonine using bacteria belonging to the genus Escherichia

(76) Inventors: Valery Zavenovich Akhverdian, Moscow (RU); Ekaterina Alekseevna Savrasova, Moscow (RU); Alla Markovna Kaplan, Moscow (RU); Andrey Olegovich Lobanov, Moscow (RU); Yuri Ivanovich Kozlov, Moscow (RU)

Correspondence Address: AJINOMOTO CORPORATE SERVICES, LLC, INTELLECTUAL PROPERTY DEPARTMENT, 1120 CONNECTICUT AVE., N.W., WASHINGTON, DC, 20036, US

(21) Appl. No.: 10/673,786

(22) Filed: Sep. 30, 2003

Related U.S. Application Data
(63) Continuation of application No. PCT/JP03/02067, filed on Feb. 25, 2003.

(30) Foreign Application Priority Data
Feb. 27, 2002 (RU) 2002104983

(51) Int. Cl. C12P 13/04; C12N 1/21

(52) U.S. Cl. 435/252.33; 435/106

(57) ABSTRACT

There is disclosed a method for producing L-threonine using bacterium belonging to the genus Escherichia wherein the bacterium has L-theonine productivity and has been modified to enhance an activity of aspartate aminotransferase.

This application is a continuation of application PCT/JP03/02067, filed Feb. 27, 2003. All documents cited herein, as well as the foreign priority document, Russia 2002104983, filed Feb. 27, 2002, are hereby incorporated by reference.

### BACKGROUND OF THE INVENTION

1. Field of the Invention

The present invention relates to biotechnology, specifically to a method for producing L-amino acids by fermentation and more specifically to a gene derived from bacterium Escherichia coli. The gene is useful for improvement of L-amino acid productivity, for example, L-threonine.

2. Brief Description of the Related Art

Conventionally, L-amino acids have been industrially produced by fermentation methods utilizing strains of microorganisms obtained from natural sources, or mutants of the same especially modified to enhance L-amino acid productivity.

One example of a-method used to enhance L-amino acid productivity is amplification of biosynthetic genes by transformation of a microorganism by recombinant DNA (see, for example, U.S. Pat. No. 4,278,765). These techniques are based on increasing the activity of the enzymes involved in amino acid biosynthesis and/or desensitizing the target enzymes to the feedback inhibition by the resulting L-amino acid or its by-products (see, for example, Japanese Laid-open application No56-18596 (1981), WO 95/16042 or U.S. Pat. Nos. 5,661,012 and 6,040,160).

Various strains used for production of L-threonine by fermentation are known. There are strains with increased activities of the enzymes involved in L-threonine biosynthesis (U.S. Pat. Nos. 5,175,107; 5,661,012; 5,705,371; 5,939,307; EP0219027), strains resistant to some chemicals such as L-threonine and its analogs (WO0114525A1, EP301572A2, U.S. Pat. No. 5,376,538), strains with the target enzymes desensitized to the feedback inhibition by the resulting L-amino acid or its by-products (U.S. Pat Nos. 5,175,107; 5,661,012), strains with inactivated threonine degradation enzymes (U.S. Pat. Nos. 5,939,307; 6,297,031).

The known threonine producing strain VKPM B-3996 (U.S. Pat. Nos. 5,175,107, and 5,705,371) is the best threonine producer at present. For construction of the strain VKPM B-3996 several mutations and a plasmid described below were introduced in the parent strain E. coli K-12 (VKPM B-7). Mutant thrA gene (mutation thrA442) encodes aspartokinase homoserine dehydrogenase I resistant to feedback inhibition by threonine. Mutant ilvA gene (mutation ilvA442) encodes threonine deaminase with low activity leading to a low rate of isoleucine biosynthesis and to a leaky phenotype of isoleucine starvation. In bacteria with ilvA442 mutation, transcription of thrABC operon isn't repressed by isoleucine and therefore is very efficient for threonine production. Inactivation of tdh gene leads to prevention of the threonine degradation. The genetic determinant of saccharose assimilation (scrKYABR genes) was transferred to said strain. To increase expression of genes controlling threonine biosynthesis, plasmid pVIC40 containing mutant threonine operon thrA442BC was introduced in the intermediate strain TDH6. The amount of L-threonine accumulated during fermentation of the strain reaches up to 85 g/l .

The present inventors obtained, with respect to E. coli K-12, a mutant having a mutation, thrR (herein referred to as rhtA23) that is concerned in resistance to high concentrations of threonine or homoserine in a minimal medium (Astaurova, O. B. et al., Appl. Bioch. And Microbiol., 21, 611-616 (1985)). The mutation improved the production of L-threonine (SU Patent No. 974817), homoserine and glutamate (Astaurova, O. B. et al., Appl. Bioch. And Microbiol., 27, 556-561, 1991, EP 1013765 A) by the respective E. coli producing strain, such as the strain VKPM-3996. Furthermore, the present inventors have revealed that the rhtA gene exists at 18 min on E. coli chromosome close to the glnHPQ operon that encodes components of the glutamine transport system, and that the rhtA gene is identical to ORF1 (ybiF gene, numbers 764 to 1651 in the GenBank accession number AAA218541, gi:440181), located between pexB and ompX genes. The unit expressing a protein encoded by the ORF1 has been designated as rhtA (rht: resistance to homoserine and threonine) gene. Also, the present inventors have found that the rhtA23 mutation is an A-for-G substitution at position—1 with respect to the ATG start codon (ABSTRACTS of 17^{th} International Congress of Biochemistry and Molecular Biology in conjugation with 1997 Annual Meeting of the American Society for Biochemistry and Molecular Biology, San Francisco, Calif. Aug. 24-29, 1997, abstract No. 457, EP 1013765 A).

Under conditions for studying the mainstream threonine biosynthetic pathway and optimizing to a great extent, the further improvement of threonine-producing strain could be done by supplementing bacterium with increased amount of distant precursors of threonine such as aspartate.

It is known that aspartate is a donor of carbon for synthesis of the amino acids of the aspartate family (threonine, methionine, lysine), and diaminopimelate (a compound constituent of the bacterial cell wall). These syntheses are performed by a complex pathway with several branch points and an extremely sensitive regulatory scheme. At the branch point of aspartate, aspartate semialdehyde, homoserine, there are as many isozymes as there are amino acids deriving from this biosynthetic step. The aspartokinase homoserine dehydrogenase I encoded by (part of thrABC operon) performs first and third reactions of threonine biosynthesis. Threonine and isoleucine regulate the expression of aspartokinase homoserine dehydrogenase I, and threonine inhibits both activities to catalyze the above-mentioned reactions (Escherichia coli and Salmonella, Second Edition, Editor in Chief: F. C. Neidhardt, ASM Press, Washington D.C., 1996).

Two genes are involved in the formation of aspartate—aspartate aminotransferase (aspartate transaminase) encoded by aspC gene, and aspartase, which is a product of aspA gene. Aspartate aminotransferase converts oxaloacetate to aspartate. Aspartase converts fumarate to aspartate.

The effect of amplification of aspC gene on production of L-lysine—an amino acid of aspartate family—is disclosed. Amplification of aspC gene was used for L-lysine production by E.coli (U.S. Pat. No. 6,040,160). Coryneform bacteria harboring an aspartokinase and enhanced DNA sequence coding for several enzymes including aspartate aminotransferase was used for L-lysine production (U.S. Pat. No. 6,004,773).

It was noticed that aspartate aminotransferase could be useful for production of L-threonine and L-lysine by coryneform bacteria (U.S. Pat. No. 4,980,285).

To date there is no report of using the bacterium belonging to the genus Escherichia with enhanced aspartate aminotransferase activity for production of L-threonine.

### SUMMARY OF THE INVENTION

An object of present invention is to enhance the productivity of L-threonine-producing strains and to provide a method for producing L-threonine using these strains.

This aim was achieved by finding that the aspC gene encoding aspartate aminotransferase cloned on a low copy vector can enhance L-threonine production. Thus the present invention has been completed.

It is an object of the invention to provide an L-threonine-producing bacterium belonging to the genus Escherichia, wherein the bacterium has been modified to enhance an activity of aspartate aminotransferase.

It is a further object of the invention to provide a bacterium wherein the activity of aspartate aminotransferase is enhanced by increasing expression of an aspartate aminotransferase gene.

It is a further object of the invention to provide a bacterium wherein the activity of aspartate aminotransferase is increased by increasing the copy number of the aspartate aminotransferase gene, or modifying an expression control sequence of the gene so that the expression of the gene is enhanced.

It is a further object of the invention to provide a bacterium as described in the preceeding paragraphs wherein the copy number of the aspartate aminotransferase gene is increased by transformation of the bacterium with a low copy vector containing the gene.

It is a further object of the invention to provide a bacterium as described in the preceeding paragraphs wherein the aspartate aminotransferase gene is originated from a bacterium belonging to the genus Escherichia.

It is a further object of the invention to provide the bacterium as described in the preceeding paragraphs, wherein the aspartate aminotransferase gene encodes the following protein (A) or (B):

(A) a protein, which comprises the amino acid sequence shown in SEQ ID NO: 2;

(B) a protein which comprises an amino acid sequence including deletion, substitution, insertion or addition of one or several amino acids in the amino acid sequence shown in SEQ ID NO: 2, and which has an activity of aspartate aminotransferase.

It is a further object of the invention to provide the bacterium as described in the preceeding paragraphs, wherein the aspartate aminotransferase gene comprises the following DNA (a) or (b):

(a) a DNA which comprises a nucleotide sequence of the nucleotides 1 to 1196 in SEQ ID NO: 1; or

(b) a DNA which is hybridizable with a nucleotide sequence of the nucleotides 1-1196 in SEQ ID NO: 1, or a probe which can be prepared from the nucleotide sequence under the stringent conditions and codes for a protein having an activity of aspartate aminotransferase.

It is a further object of the invention to provide the bacterium as described in the preceeding paragraphs, wherein the stringent conditions comprise washing at 60° C. and at a salt concentration corresponding to 1×SSC and 0.1% SDS.

It is a further object of the invention to provide the bacterium as described in the preceeding paragraphs, wherein the bacterium has been further modified to enhance expression of one or more genes selected from the group consisting of

the mutant thrA gene which codes for aspartokinase homoserine dehydrogenase I resistant to feed back inhibition by threonine;

the thrB gene which codes for homoserine kinase;

the thrC gene which codes for threonine synthase;

the rhtA gene, which codes for putative transmembrane protein.

It is a further object of the invention to provide the bacterium as described in the preceeding paragraphs, wherein the bacterium has been modified to increase expression of the mutant thrA gene, the thrB gene, the thrC gene and the rhta gene.

It is a further object of the invention to provide a method for producing L-threonine, which comprises cultivating the bacterium as described in the preceeding paragraphs in a culture medium to produce and accumulate L-threonine in the culture medium, and collecting the L-threonine from the culture medium.

Still other objects, features and attendant advantages of the present invention will become apparent to those skilled in the art from a reading of the following detailed description of the embodiments and examples.

### DESCRIPTION OF THE PREFERRED EMBODIMENTS

The bacterium of the present invention is an L-threonine-producing bacterium belonging to the genus Escherichia, wherein the bacterium has been modified to enhance an activity of aspartate aminotransferase.

The bacterium belonging to the genus Escherichia that can be used in the present invention includes, but is not particularly limited to, bacteria described by Neidhardt, F. C. et al. (Escherichia coli and Salmonella typhimurium, American Society for Microbiology, Washington D.C., 1208, Table 1).

In the present invention, “L-threonine-producing bacterium” means a bacterium which has an ability to accumulate L-threonine in a medium, when the bacterium of the present invention is cultured in the medium. The L-threonine-producing ability may be imparted or enhanced by breeding.

The phrase “activity of aspartate aminotransferase” means activity to catalyze the reaction of formation the aspartate from oxaloacetate and L-glutamate with release of α-ketoglutarate using pyridoxal 5′-phosphate.

The phrase “modified to enhance an activity of aspartate aminotransferase” means that the activity per cell has become higher than that of a non-modified strain, for example, a wild-type strain. Examples include, but are not limited to, a case where number of aspartate aminotransferase molecules per cell increases, or a case where specific activity per aspartate aminotransferase molecule increases, and so forth. Furthermore, a wild-type strain that might serve as a comparison includes, but is not limited to, the Escherichia coli K-12. As a result of enhancement of intracellular activity of aspartate aminotransferase, the amount of L-threonine accumulation in a medium may increase.

Enhancement of aspartate aminotransferase activity in a bacterial cell can be attained by enhancing the expression of a gene coding for aspartate aminotransferase. Any of genes derived from bacteria belonging to the genus Escherichia and genes derived from other bacteria such as coryneform bacteria can be used as the aspartate aminotransferase gene. Among these, genes derived from bacteria belonging to the genus Escherichia are preferred.

As the gene coding for aspartate aminotransferase of Escherichia coli, aspC has already been elucidated (nucleotide numbers 983742 to 984932 in the sequence of GenBank accession NC_{—}000913.1, gi: 16128895). Therefore, aspC gene can be obtained by PCR (polymerase chain reaction; refer to White, T. J. et al., Trends Genet., 5, 185 (1989)) utilizing primers prepared based on the nucleotide sequence of the gene. Genes coding for aspartate aminotransferase of other microorganisms can be obtained in a similar manner.

The aspC gene originated from Escherichia coli is exemplified by a DNA which encodes the following protein (A) or (B):

(A) a protein, which comprises the amino acid sequence shown in SEQ ID NO: 2;

(B) a protein which comprises an amino acid sequence including deletion, substitution, insertion or addition of one or several amino acids in the amino acid sequence shown in SEQ ID NO: 2, and which has an activity of aspartate aminotransferase.

The number of “several” amino acids differs depending on the position or the type of amino acid residues in the three dimensional structure of the protein. It may be 2 to 30, preferably 2 to 15, and more preferably 2 to 5 of the protein (A). This is because some amino acids have high homology to one another and the difference in such an amino acid does not greatly affect the three dimensional structure of the protein and its activity. Therefore, the protein (B) may be one which has homology of not less than 30 to 50%, preferably 50 to 70% with respect to the entire amino acid sequence of aspartate aminotransferase, and which has the activity of aspartate aminotransferase.

The DNA which codes for substantially the same protein as the aspartate aminotransferase described above may be obtained, for example, by modifying the nucleotide sequence of DNA coding for aspartate aminotransferase (SEQ ID NO: 1), for example, by means of site-directed mutagenesis so that one or more amino acid residues at a specified site involve deletion, substitution, insertion, or addition. DNA modified as described above may be obtained by the conventionally known mutation treatment. Such treatment includes treatment of the DNA coding for proteins of present invention with hydroxylamine, or treatment of the bacterium harboring the DNA with UV irradiation or a reagent such as N-methyl-N′-nitro-N-nitrosoguanidine or nitrous acid.

A DNA coding for substantially the same protein as aspartate aminotransferase can be obtained by expressing a DNA having such a mutation as described above in an appropriate cell, and investigating the activity of an expressed product. A DNA coding for substantially the same protein as aspartate aminotransferase can also be obtained by isolating a DNA that is hybridizable with a probe having a nucleotide sequence comprising, for example, the nucleotide sequence shown in SEQ ID NO: 1, under the stringent conditions, and codes for a protein having the aspartate aminotransferase activity, from DNA coding for aspartate aminotransferase having a mutation or from a cell harboring it. The “stringent conditions” referred to herein is a condition under which so-called specific hybrid is formed, and non-specific hybrid is not formed. It is difficult to clearly express this condition by using any numerical value. However, for example, the stringent conditions are exemplified by a condition under which DNAs having high homology, for example, DNAs having homology of not less than 50% are hybridized with each other, but DNAs having homology lower than the above are not hybridized with each other. Alternatively, the stringent conditions are exemplified by a condition under which DNAs are hybridized with each other at a salt concentration corresponding to an ordinary condition of washing in Southern hybridization, i.e., 1×SSC, 0.1% SDS, preferably 0.1×SSC, 0.1% SDS, at 60° C.

A partial sequence of the nucleotide sequence of SEQ ID NO: 1 can also be used as a probe. Such a probe may be prepared by PCR using oligonucleotides produced based on the nucleotide sequence of SEQ ID NO: 1 as primers, and a DNA fragment containing the nucleotide sequence of SEQ ID NO: 1 as a template. When a DNA fragment in a length of about 300 bp is used as the probe, the conditions of washing for the hybridization consist of, for example, 50° C., 2×SSC and 0.1% SDS.

The substitution, deletion, insertion, or addition of nucleotide as described above also includes mutation, which naturally occurs (mutant or variant), for example, on the basis of the individual difference or the difference in species or genus of bacterium, which harbors aspartate aminotransferase.

Transformation of a bacterium with DNA coding for protein means introduction of the DNA into bacterium cell, for example, by conventional methods to increase expression of the gene coding for the protein of present invention and to enhance the activity of the protein in the bacterial cell.

Methods of enhancing gene expression include increasing the gene copy number. Introduction of a gene into a vector that is able to function in a bacterium belonging to the genus Escherichia increases copy number of the gene. For such purposes, low copy vectors can be preferably used. The low-copy vector is exemplified by pSC101, pMW118, pMW119 and the like. As the method of transformation, any known method that has hitherto been reported can be employed. For instance, a method of treating recipient cells with calcium chloride so as to increase the permeability of DNA, which has been reported for Escherichia coli K-12 (Mandel, M. and Higa, A., J. Mol. Biol., 53, 159 (1970)), may be used.

Enhancing gene expression can also be achieved by introduction of multiple copies of the gene into bacterial chromosome by, for example, methods of homologous recombination, or the like.

On the other hand, enhancing gene expression can also be achieved by placing the DNA of the present invention under the control of a potent promoter. For example, lac promoter, trp promoter, trc promoter, P_{R}, P_{L} promoters of lambda phage are known as potent promoters. Using the potent promoter can be combined with the multiplication of gene copies.

Alternatively, a promoter can be enhanced by, for example, introducing a mutation into the promoter to increase a transcription level of a gene located downstream of the promoter. Furthermore, it is known that substitution of several nucleotides in the spacer between ribosome binding site (RBS) and start codon and especially the sequences immediately upstream of the start codon profoundly affect the mRNA translatability. For example, a 20-fold range in the expression levels was found depending on the nature of the three nucleotides preceding the start codon (Gold et al., Annu. Rev. Microbiol., 35, 365-403, 1981;, Hui etal., EMBO J., 3, 623-629, 1984). Earlier, the authors of present invention showed the rhtA23 mutation is an A-for-G substitution at position—1 with respect to the ATG start codon (ABSTRACTS of 17^{th} International Congress of Biochemistry and Molecular Biology in conjugation with 1997 Annual Meeting of the American Society for Biochemistry and Molecular Biology, San Francisco, Calif. Aug. 24-29, 1997, abstract No. 457). Therefore, it may be suggested that rhtA23 mutation enhances the rhtA gene expression and, as a consequence, increases the level of resistance to threonine, homoserine and some other substances transported out of cells.

Moreover, it is also possible to introduce nucleotide substitution into a promoter region of the aspartate aminotransferase gene on the bacterial chromosome so that it should be modified into a stronger one. Alteration of the expression control sequence can be performed, for example, in the same manner as the gene substitution using a temperature sensitive plasmid, as disclosed in International Patent Publication WO00/18935 and Japanese Patent Publication No. 1-215280.

Increasing the copy number of aspartate aminotransferase gene can also be achieved by introducing multiple copies of the aspartate aminotransferase gene into chromosomal DNA of bacterium. In order to introduce multiple copies of the aspartate aminotransferase gene into the bacterial chromosome, homologous recombination is carried out by using a sequence whose multiple copies exist in the chromosomal DNA as targets. As sequences whose multiple copies exist in the chromosomal DNA, repetitive DNA, inverted repeats existing at the end of a transposable element can be used. Also, as disclosed in Japanese Patent Laid-open No. 2-109985, it is possible to incorporate the aspartate aminotransferase gene into transposon, and allow it to be transferred to introduce multiple copies of the gene into the chromosomal DNA.

Methods for preparation of plasmid DNA, digestion and ligation of DNA, transformation, selection of an oligonucleotide as a primer and the like may be ordinary methods well known to one skilled in the art. These methods are described, for instance, in Sambrook, J., Fritsch, E. F., and Maniatis, T., “Molecular Cloning A Laboratory Manual, Second Edition”, Cold Spring Harbor Laboratory Press (1989).

The bacterium of the present invention can be obtained by introduction of the aforementioned DNAs into bacterium inherently having the ability to produce L-threonine. Alternatively, the bacterium of present invention can be obtained by imparting the ability to produce L-threonine to the bacterium already harboring the DNAs.

As a parent strain which is to be enhanced in activity of the aspartate aminotransferase encoded by aspC gene, the threonine producing bacteria belonging to the genus Escherichia such as E. coli strain VKPM B-3996 (U.S. Pat. No. 5,175,107, U.S. Pat. No. 5,705,371), E. coli strain NRRL-21593 (U.S. Pat. No. 5,939,307), E. coli strain FERM BP-3756 (U.S. Pat. No. 5,474,918), E. coli strains FERM BP-3519 and FERM BP-3520 (U.S. Pat. No. 5,376,538), E. coli strain MG442 (Gusyatiner et al., Genetika (in Russian), 14, 947-956 (1978)), E. coli strains VL643 and VL2055 (EP 1149911 A) and the like may be used.

The strain B-3996 is deficient in thrC gene and is sucrose-assimilative, in which ilvA gene has a leaky mutation. The strain has a mutation in rhtA gene, which confers resistance to high concentration of threonine or homoserine. The strain B-3996 harbors the plasmid pVIC40 which had been obtained by inserting thrA*BC operon including mutant thrA gene encoding aspartokinase homoserine dehydrogenase I which is substantially desensitized feedback inhibition by threonine into RSF1010-derived vector. The strain B-3996 was deposited on Nov. 19, 1987 in All-Union Scientific Center of Antibiotics (Nagatinskaya Street 3-A,113105 Moscow,Russian Federation) under the accession number RIA 1867. The strain was also deposited in Russian National Collection of Industrial Microorganisms (VKPM) (Dorozhny proezd. 1, Moscow 113545, Russian Federation) under the accession number B-3996.

The bacterium of the present invention is preferably further modified to enhance expression of one or more of the following genes as well as aspC gene:

the mutant thrA gene which codes for aspartokinase homoserine dehydrogenase I resistant to feed back inhibition by threonine;

the thrB gene which codes for homoserine kinase;

the thrC gene which codes for threonine synthase;

Another preferred embodiment of the bacterium is modified to enhance the rhtA gene, which codes for putative transmembrane protein in addition to enhancement of aspC gene. The most preferred embodiment of the bacterium is modified to increase expression amount of the aspC gene, the mutant thrA gene, the thrB gene, the thrC gene and the rhtA gene.

The method for producing L-threonine of the present invention comprises the steps of cultivating the bacterium of the present invention in a culture medium, to allow L-threonine to be produced and accumulated in the culture medium, and collecting L-threonine from the culture medium.

In the present invention, the cultivation, the collection and purification of L-amino acid from the medium and the like may be performed in a manner similar to the conventional fermentation method wherein an amino acid is produced using a microorganism.

The medium used for culture may be either a synthetic medium or a natural medium, so long as the medium includes a carbon source and a nitrogen source and minerals and, if necessary, appropriate amounts of nutrients which the microorganism requires for growth. The carbon source may include various carbohydrates such as glucose and sucrose, and various organic acids. Depending on the mode of assimilation of the used microorganism, alcohol including ethanol and glycerol may be used. As the nitrogen source, various ammonium salts such as ammonia and ammonium sulfate, other nitrogen compounds such as amines, a natural nitrogen source such as peptone, soybean-hydrolysate, and digested fermentative microorganism can be used. As minerals, potassium monophosphate, magnesium sulfate, sodium chloride, ferrous sulfate, manganese sulfate, calcium chloride, and the like can be used. As vitamins, thiamine, yeast extract and the like can be used.

The cultivation is performed preferably under aerobic conditions such as a shaking culture, and stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Typically, a 1 to 5-day cultivation leads to the accumulation of the target L-amino acid in the liquid medium.

After cultivation, solids such as cells can be removed from the liquid medium by centrifugation or membrane filtration, and then L-threonine can be collected and purified by ion-exchange, concentration and crystallization methods.

The present invention will be more concretely explained below with reference to following Examples, which are intended to be illustrative only and are not intended to limit the scope of the invention as defined by the appended claims.

### EXAMPLES

### Example 1

### Cloning of aspC Gene from E. coli into pMW119 Vector

The aspC gene was obtained from chromosomal DNA of the E. coli strain K-12 by PCR using primers shown in SEQ ID NOs: 3 and 4. The obtained DNA fragment was treated with PvuII and EcoRI restrictases and ligated to the stable low copy plasmid pMW119 (replicon pSC101) previously treated with HincII and EcoRI restrictases under control of P_{lac} promoter. Thus, the pMW-P_{lac}-aspC plasmid was obtained.

Also, aspC gene was placed under control of the strong P_{R} promoter of the phage lambda instead of P_{lac} promoter. A DNA duplex containing P_{R} promoter was formed using chemically synthesized 5′-phosphorylated oligonucleotides shown in the SEQ ID Nos: 5 and 6. Then, the DNA duplex was ligated to the pMW-P_{lac}-aspC plasmid previously treated with PvuII and HindIII restrictases. Thus the plasmid pM-P_{R}-aspC was constructed.

Non-regulated high level of aspC gene expression could be achieved using these plasmids. The plasmids pMW-P_{lac}-aspC and pM-P_{R}-aspC are compatible with plasmid pVIC40 (replicon pRSF1010), therefore two plasmids pVIC40 and pMW-P_{lac}-aspC or pVIC40 and pM-P_{R}-aspC could be maintained in the bacteria simultaneously. Each of the pMW-P_{lac}-aspC and pM-P_{R}-aspC plasmid was introduced into streptomycin-resistant threonine producer E. coli strain B-3996 (U.S. Pat. No. 5,175,107). Thus the strains B-3996(pMW-P_{lac}-aspC) and B-3996(pM-P_{R}-aspC) were obtained.

### Example 2

### Effect of the aspC Gene Amplification on Threonine Production

The E. coli strain VKPM-3996(pM-P_{R}-aspC) was grown for 18-24 hours at 37° C. on L-agar plates containing streptomycin (100 μg/ml). Then one loop of the cells was transferred to 50 ml of L-broth of the following composition: trypton—10 g/l, yeast extract—5 g/l, NaCl—5 g/l. The cells (50 ml, OD₅₄₀-2 o.u.) grown at 37° C. within 5 hours on shaker (240 rpm) was used for seeding 450 ml of the medium for fermentation. The batch fermentation was performed in laboratory fermenters having a capacity of 1.01 under aeration (1/1 vvm) with stirring at a speed of 1200 rpm at 37° C. The pH value was maintained automatically at 6.6 using 8% ammonia liquor. The results are presented in Table 1.

The composition of the fermentation medium (g/l):

| | |
|---|---|
| Sucrose | 100.0 |
| NH₄Cl | 1.75 |
| KH₂PO₄ | 1.0 |
| MgSO₄ × 7H₂O | 0.8 |
| FeSO₄ × 7H₂O | 0.01 |
| MnSO₄ × 5H₂O | 0.01 |
| Mameno(TN) | 0.15 |
| Betaine | 1.0 |
| L-isoleucine | 0.2 |

Sucrose and magnesium sulfate are sterilized separately. pH is adjusted to 6.6.

TABLE 1

| | Additional | | | |
|---|---|---|---|---|
| Strain | aspC gene | Time, hours | OD₅₄₀ | Threonine, g/l |
| B-3996 | − | 19.1 | 34.6 | 45.0 |
| | | 18.4 | 33.8 | 43.8 |
| B-3996 | + | 17.5 | 29.8 | 45.2 |
| (pMW-P_{lac}- | | 18.5 | 30.6 | 45.8 |
| aspC) | | 18.5 | 30.8 | 45.9 |
| | | 18.3 | 30.0 | 46.2 |
| | | 18.2 ± 0.5 | 30.3 ± 0.5 | 45.9 ± 0.4 |
| B-3996 | + | 16.0 | 32.0 | 45.4 |
| (pM-P_{R}- | | 18.0 | 30.8 | 46.5 |
| aspC) | | 17.8 | 29.4 | 45.8 |
| | | 18.3 | 30.0 | 45.2 |
| | | 17.9 ± 1.0 | 30.4 ± 1.1 | 45.6 ± 0.6 |

While the invention has been described in detail with reference to preferred embodiments thereof, it will be apparent to one skilled in the art that various changes can be made, and equivalents employed, without departing from the scope of the invention.

What is claimed is:

1. An L-threonine-producing bacterium belonging to the genus Escherichia, wherein the bacterium has been modified to enhance an activity of aspartate aminotransferase.

2. The bacterium of claim 1, wherein said activity of aspartate aminotransferase is enhanced by increasing the expression of an aspartate aminotransferase gene.

3. The bacterium of claim 1, wherein said activity of aspartate aminotransferase is increased by a method selected from the group consisting of increasing the copy number of the aspartate aminotransferase gene, and modifying an expression control sequence of said gene so that the expression of said gene is enhanced.

4. The bacterium according to claim 3, wherein said activity of aspartate aminotransferase is increased by increasing the copy number of the aspartate aminotransferase gene.

5. The bacterium of claim 4, wherein the copy number is increased by transformation of said bacterium with a low copy vector containing said gene.

6. The bacterium of claim 2, wherein said aspartate aminotransferase gene is originated from a bacterium belonging to the genus Escherichia.

7. The bacterium of claim 6, wherein said aspartate aminotransferase gene encodes a protein selected from the group consisting of:
(A) a protein comprising the amino acid sequence shown in SEQ ID NO: 2; and
(B) a protein comprising an amino acid sequence including deletion, substitution, insertion or addition of one or several amino acids in the amino acid sequence shown in SEQ ID NO: 2, and which has an activity of aspartate aminotransferase.

8. The bacterium of claim 6, wherein said aspartate aminotransferase gene comprises DNA selected from the group consisting of:
(a) a DNA comprising a nucleotide sequence of the nucleotides 1 to 1196 in SEQ ID NO: 1; and
(b) a DNA which is hybridizable with a nucleotide sequence of the nucleotides 1-1196 in SEQ ID NO: 1 or a probe which can be prepared from said nucleotide sequence under stringent conditions, and codes for a protein having an activity of aspartate aminotransferase.

9. The bacterium of claim 8, wherein said stringent conditions are washing at 60° C. and at a salt concentration corresponding to 1×SSC and 0.1% SDS.

10. The bacterium of claim 2, wherein said bacterium has been further modified to enhance expression of one or more genes selected from the group consisting of
the mutant thrA gene which codes for aspartokinase homoserine dehydrogenase I resistant to feed back inhibition by threonine;
the thrB gene, which codes for homoserine kinase;
the thrC gene, which codes for threonine synthase; and
the rhta gene, which codes for putative transmembrane protein.

11. The bacterium of claim 10, wherein said bacterium has been modified to increase expression of said mutant thrA gene, said thrB gene, said thrC gene and said rhtA gene.

12. A method for producing L-threonine which comprises cultivating the bacterium of claim 1 in a culture medium to produce and accumulate L-threonine in the culture medium, and collecting the L-threonine from the culture medium.
